(19) United States
(12) Patent Application Publication
(10) Pub. No.: US 2004/0040515 A1
(43) Pub. Date: Mar. 4, 2004

(54) Method and apparatus for determining the sex of a fertilized egg

(75) Inventor: Ryosuke Taniguchi, Nagasaki-ken (JP)

Correspondence Address: FRISHAUF, HOLTZ, GOODMAN & CHICK, PC, 767 THIRD AVENUE, 25TH FLOOR, NEW YORK, NY, 10017-2023, US

(73) Assignees: KABUSIKI KAISYA HORIUCHI, Kurume-shi (JP); TOWA SANGYO KABUSIKI KAISYA, Nohgata-shi (JP)

(21) Appl. No.: 10/232,872

(22) Filed: Aug. 30, 2002

(51) Int. Cl. A01K 31/19; A01K 41/00; G06F 19/00; G01N 33/48; G01N 33/50

(52) U.S. Cl. 119/300; 702/19

(57) ABSTRACT

The present invention provides a practical, reliable, high-speed, automated method for determining the sex of a fertilized chicken egg using threshold values obtained by measuring the shapes of a plurality of eggs of a given variety of chicken, determining the sex of the chicks actually hatched from the eggs, correlating the shape of the egg to the sex of the hatchling and extracting distinctive common features of the egg shape (regardless of size) to accurately and reliably determine the sex of fertilized eggs.

### FIELD OF THE INVENTION

The present invention relates to an improved method and apparatus for determining the sex of a fertilized chicken egg, and more particularly, to an improved method and apparatus for determining the sex of a fertilized chicken egg in which threshold values obtained by cataloging the various shapes of chicken eggs of any given variety of chicken and extracting the features in common to all such eggs is used to computer-process the shape of any egg to be sexed.

### BACKGROUND OF THE INVENTION

Among birds such as chickens, which include varieties raised mainly for their eggs such as the White Leghorn, only the egg-producing females are useful. By contrast, among chickens raised for their meat, both the males and the females are useful, although males and females mature at different rates and it is therefore more efficient to raise them separately. In the case of both types of the above-described varieties of chickens, for these and other reasons the chicks are sexed 2-3 days after they are hatched in order to determine what sex they are.

Three methods are currently used to determine the sex of the hatchlings: (a) by hand, (b) by machine or (c) by utilizing sex-controlled inheritance (that is, sex-linked inheritance) characteristics.

All three of the above-described conventional methods involve sexing a hatched chick, so unless the egg is hatched the sex of the chick to be hatched cannot be determined. As a result, in the case of the White Leghorn chickens raised to produce eggs, for example, the time and expense of hatching eggs containing male chicks is wasted. In addition, in the case of such egg-producing varieties of chickens, those chicks found to be male are destroyed, which is undesirable from the viewpoint that life in all its forms should be respected.

If it were possible to reliably sex the chicks before they are hatched, that is, while still in the egg, the above-described wastage and dilemma could be avoided. Prior to hatching, for example, the males could be used for food or for the production of vaccines.

A publicly known method of determining the sex of the chick in the egg stage involves comparing the shape of the projected curve of the egg at specific points. In this technique, a profile projector is used to take a blown-up profile of the laid egg for each hen, after which the eggs are allowed to hatch, the hatchlings are sexed, and the shapes of the eggs are categorized according to the sex of the resulting chick. This process is repeated over a certain period of time until a reference range is established for each hen, after which reference profiles are produced. Thereafter, the shape of a laid egg to be sexed is then compared to the reference profiles in order to determine the sex of the unhatched chick.

However, the above-described conventional chicken egg sexing technique has the following drawbacks.

(a) It is commercially impractical to get reference profiles for each hen, because a lot of chickens are bred in the poultry farming business.

(b) The technique relies on the human eye to compare the wide end of the egg to be sexed against the reference profile established for that hen, and as such is not entirely reliable.

(c) The work of producing the reference profiles for sexing the eggs involves the above-described steps, so it is a relatively lengthy process.

### SUMMARY OF THE INVENTION

Accordingly, the inventors of the present invention conducted a number of experiments in an effort to develop a practical, reliable method for determining the sex of an unhatched chicken egg. Specifically, the inventors collected a number of fertilized eggs from several hens of the same variety, measured the shapes of the eggs, hatched the eggs, and had a professional determine the sex of the chicks hatched from the shape-measured eggs. The inventors then used statistical means such as scatter diagrams and the like to analyze the relation between the sex of the chicks and the shapes of the eggs from which they were hatched.

Based on the results of the analysis of the relation between egg shape and hatchling sex, the inventors were able to identify several distinctive features common to all eggs obtained from the same variety of chickens without regard to size, and were therefore able to determine that it is possible to determine the sex of the unhatched egg from the shape of the egg itself. In addition, the inventors were able to identify significant differences between varieties of chickens, and to show that these differences were not dependent on the size of individual hens.

The present invention is based on the above-described work, and has the following objects. The first object is to provide a more practical method and apparatus for determining the sex of a fertilized chicken egg. The second is to provide a more reliable method and apparatus for determining the sex of a fertilized chicken egg. The third is to provide a method for determining the sex of a fertilized chicken egg from the general features of the egg by variety of chicken. The fourth object is to establish standards used to determine the sex of the fertilized chicken egg and to use those standards to provide a quick technique for determining the sex of the fertilized egg.

The above-described objects of the present invention are achieved by a method in which measurements are made on a plurality of eggs obtained form chickens of the same variety. The measured eggs are then hatched and the sex of the hatchlings determined to obtain the threshold value that represents the sex of the hatchlings. The threshold value of hatchlings and the measured or computed numerical data on the shape of the eggs are then correlated using statistical means such as scatter diagrams and the like. The results of that analysis are then used to extract distinctive, numerical differences in the shapes of the fertilized chicken eggs. In addition, by finding features that differ depending on the variety of chicken, for example, the method can be used for a plurality of hens in common, provided the hens are of the same variety.

Other objects, features and advantages of the present invention besides those discussed above shall be apparent to those skilled in the art from the description of a preferred embodiment of the invention which follows. In the description, reference is made to accompanying drawings, which form a part thereof, and which illustrate examples of the invention. Such examples, however, are not exhaustive of the various embodiments of the invention, and therefore reference is made to the claims that follow the description for determining the scope of the invention.

### BRIEF DESCRIPTION OF THE DRAWINGS

The invention will be better understood and its numerous objects and advantages will become more apparent to those skilled in the art by reference to the following drawings, in conjunction with the accompanying specification, in which:

FIG. 1 is a flow chart showing steps in the construction of an algorithm for determining the sex of a fertilized chicken egg according to the present invention;

FIG. 2 is a flow chart showing an example of the method for determining the sex of a fertilized chicken egg according to the present invention;

FIG. 3 is a flow chart showing specific steps in the sex determination portion of the process shown in FIG. 2;

FIG. 4 is a diagram showing a profile of a chicken egg and the major dimensions and coordinates thereof;

FIG. 5 is a graph plotting results of calculations performed using a sexing equation (y/x) on the chicken eggs to be sexed;

FIG. 6 is a graph plotting results of calculations performed using a sexing equation (L/(y/2)) on the chicken eggs to be sexed;

FIG. 7 is a diagram illustrating a sexing system including an apparatus for determining the sex of a fertilized chicken egg according to the present invention;

FIG. 8 is a diagram showing an apparatus for determining the sex of a fertilized chicken egg and a front view of an image-sensing apparatus as part of the apparatus for determining the sex of a fertilized chicken egg;

FIG. 9 is a lateral view of the image-sensing apparatus of the apparatus for determining the sex of a fertilized chicken egg shown in FIG. 8;

FIG. 10 is a plan view of the egg stand of the image-sensing apparatus;

FIG. 11 is a vertical cross-sectional view of the egg stand of the image-sensing apparatus;

FIG. 12 is a diagram illustrating one method of illuminating a chicken egg using a lighting fixture of the image-sensing apparatus;

FIG. 13 is a diagram illustrating another method of illuminating a chicken egg using a lighting fixture of the image-sensing apparatus;

FIG. 14 is a flow chart showing another example of the method for determining the sex of a fertilized chicken egg, showing specific steps in the sex determination portion of a process similar to the process shown in FIG. 1;

FIG. 15 is a graph plotting results of calculations performed using a sexing equation (y/x) on the chicken eggs to be sexed;

FIG. 16 is a graph plotting results of calculations performed using a sexing equation (L/y) on the chicken eggs to be sexed; and

FIG. 17 is a table showing the sex of the chicks hatched from the measured eggs, together with the results of calculations performed using the sexing equations.

### DETAILED DESCRIPTION OF THE PREFERRED EMBODIMENTS

Preferred embodiments of the present invention will be described in detail in accordance with the accompanying drawings.

The eggs to be sexed are not limited to chicken eggs, and may be of any type. In addition, though the varieties of chickens may be White Leghorn, silky fowl, or broiler chicken (such as Cornish game hen, for example) the invention is not limited to any particular variety.

In this specification, the term “chicken egg” refers to any fertilized chicken egg capable of being hatched. In this specification.

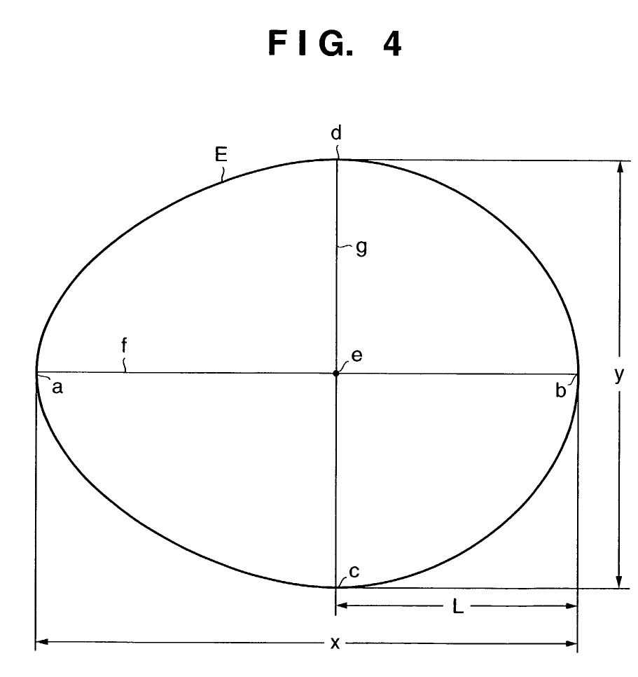

FIG. 4 is a diagram showing a profile of a chicken egg and the major dimensions and coordinates thereof. As shown in the diagram, Point a represents a coordinate of narrow end. In this specification, “A narrow end” refers to a left end a in FIG. 4 which is on a narrow end side of the contour of the egg.

Point b represents a coordinate of blunt end. In this specification, “A blunt end” refers to a right end b in FIG. 4 that is on a blunt end side of the contour of the egg.

Point c represents a coordinate of lower end. In this specification, “A bottom end” refers to a lowermost point c in FIG. 4 of the contour of the egg in FIG. 4, the point c being located on a circumference having a minor axis of the egg.

Point d represents a coordinate of upper end. In this specification, “An upper end” refers to an uppermost point d in FIG. 4 of the contour of the egg, the point d being located on a circumference having a minor axis of the egg.

In this specification, “A maximum length” of the egg refers to f which is a horizontal line segment ab connected between the wide end b and the narrow end a of the egg through the center e of the egg, and a value of the length f is represented as x.

In this specification, “A maximum width” of the egg refers to g which is a vertical line segment cd connected between the upper end d and the lower end c through the center e, and a value of the length is represented as y.

Point e represents the coordinates of a center of the egg. In this specification, “A center” refers to a point of intersection e of two lines, that is, a line which is connected between the blunt end b and the narrow end a of the egg and a line which is connected between the upper end d and the lower end c of the egg.

In this specification, “A length from a center to a blunt end (a length of wide side)” refers to a length of a line which is connected between the center e and the blunt end b, and a value of the length is represented as L.

In this specification, the term “threshold value” means a numerical threshold value obtained by statistically processing data derived from a plurality of eggs and computed according to equations required to determine the sex of the unhatched fertilized egg.

In this specification, the term “sexing value” means a numerical value obtained for each egg to be sexed, as computed from data obtained from eggs to be sexed (for example, a sensed egg image) using sexing equations required to determine the sex of the unhatched fertilized egg.

In addition, in this specification, the term “sexing” refers to the overall process, described herein, of determining the sex of an unhatched fertilized egg, based generally on the shape of the egg.

Similarly, in this specification, the term “sexing equation” means an algebraic or other mathematical formula or algorithm used to obtain a sexing value from the shape data of fertilized egg. The terms of the formula or algorithm may represent various dimensions of the shape of the egg, as described herein below.

If, for the sake of illustration, it is assumed that the length of the egg is x and the width of the egg is y, then a proportion between these two values may be expressed in terms of equations such as x/y or y/x, though such proportion is not limited to expression in terms of the above-described two equations. Similarly, “a proportion between a width of a chicken egg and a length of wide side, assuming the length is x, the width is y and the length of wide side is L, may be expressed in terms of equations such as L/y, L/(y/2), y/L or (y/2)/L, though such proportion is not limited to expression in terms of the above-described equations.

The present invention may also use an image of an egg instead of the egg itself. In that case, an image-sensing apparatus acquires the image. The image-sensing apparatus may be a digital camera, a digital video camera or the equivalent thereof. As appropriate, the present invention may also make use of a “low-reflectance background” material. Such material may be a black felt, a non-woven fabric, cloth, synthetic resin, a black-painted metal, or some other suitable material. In addition, the present invention may make use of a “cushion” material for a stand on which to hold or rest the egg during measurement or image acquisition. The cushion material may be felt, cloth, non-woven fabric, or cotton, or some other suitable material.

In the method and apparatus for determining the sex of a fertilized chicken egg according to the present invention, data on the shape of the eggs is obtained from a plurality of eggs obtained from a plurality of chickens of the same variety, after which the measured eggs are hatched and the sex of the hatchlings from these eggs is determined. Next, the shape data and the sex of hatchlings (eggs) are correlated to obtain threshold value for sexing the eggs. Further, the sex of unhatched fertilized chicken egg can be determined by comparing sexing values that are obtained by using contour data for the fertilized egg from same variety of chicken, with above threshold value. In this manner, the sex of the unhatched fertilized chicken egg can be determined on the basis of generalized features of the shapes of the eggs for a given variety of chicken, and in the processes establishing threshold value for determining the sex of a fertilized chicken egg. In addition, distinctive shape features unique to a given variety of chicken can be used to perform sexing of the eggs of that type of chicken as well, making it possible to use threshold value, in common for a plurality of hens provided they are of the same variety of chicken. Therefore, the method for determining the sex of a fertilized chicken egg of the present invention can provide a speedy, reliable and practical means of determining the sex of the unhatched chicks of a given variety of chicken.

In a method in which data on the shape of the eggs is obtained from a plurality of eggs obtained from a plurality of chickens of the same variety, after which the measured eggs are hatched and the sex of the hatchlings from these eggs are determined and the shape data and the sex of the hatchlings are correlated to obtain threshold values for determining the sex of the unhatched chicks in the eggs, quantifying the threshold values and sexing values allows the method to be computerized, which has the advantage of making it possible to automate and/or increase the speed of the sexing process.

In a method in which the length and width of a chicken egg (or an image of a chicken egg) is measured or computed and a threshold value for sexing the eggs are obtained from the measured or computed lengths and widths, and further, the length and width of an egg to be sexed is measured or computed and a sexing value obtained from that length and width, and the sexing value so obtained and the threshold value so obtained are compared and the sex of the unhatched chick in the egg is determined, by obtaining four coordinates indicating a blunt end, a narrow end, an upper end, and a lower end, respectively (since the center can be computed from the other four coordinates), it is possible to determine the sex of the unhatched egg.

In a method in which the length and width of a chicken egg (or an image of a chicken egg) is measured or computed and a threshold value for sexing the eggs are obtained from the measured or computed lengths and widths, and further, the length and width of an egg to be sexed is measured or computed and a sexing value obtained from that length and width, and the sexing value so obtained and the threshold value so obtained are compared and the sex of the unhatched chick in the egg is determined, by correcting any tilt in the image the coordinates indicating the contours of the chicken egg can be extracted more accurately, thereby providing more reliable sexing results.

In a method in which the sexing equation expresses a proportion between the length of the egg and the width of the egg, the result of the computations performed with the sexing equation is the production of a numerical sexing value indicating the rotundity of the egg. Expressing the fatness of the egg as a numerical quantity allows a comparison to be made between different eggs concerning the “fatness” of the eggs that is one important factor in determining the sex of the fertilized unhatched egg, thereby proving more reliable sexing results.

In a method in which the sexing equation expresses a proportion between a width of a chicken egg and the length of wide side of the chicken egg, the result of the computations performed using the sexing equation is a numerical value indicating the roundness of the wide side of the egg (that is, the non-pointed end of the egg). Expressing the roundness of the egg as a numerical quantity allows a comparison to be made between different eggs concerning the “roundness of the wide side of the egg” that is one important factor in determining the sex of the fertilized unhatched egg, thereby proving more reliable sexing results.

In a method in which the sexing equation represents a combination of an equation expressing a proportion between a length and a width of the chicken eggs and an equation expressing a proportion between a width of a chicken egg and the a length of wide side, the result of the computations performed using such a sexing equation enables comparisons to be made between eggs of the “rotundity” and “roundness” of the eggs that are factors important to an accurate determination of the sex of the fertilized unhatched eggs, thereby providing more reliable sexing results.

In a method in which an image of the egg to be sexed is scanned, in which the image of the egg to be measured or sexed includes maximum and minimum coordinates indicating the length of the egg and maximum and minimum coordinates indicating the width of the egg, and a scan of the image of the egg to be measured or sexed includes a portion including at least 3 coordinates when the image of the egg to be measured or sexed is scanned, in which the three coordinates including at least the maximum and minimum coordinates indicating the length of the egg, it is possible to reduce the volume of data to be processed by one half or more as compared to a full scan of the entire image, thus allowing a reduction in the load on the image processing apparatus such as a computer system, thereby providing speedier sexing results.

According to the improved apparatus for determining the sex of a fertilized chicken egg of the present invention, it is possible to sense or acquire an image of the shape of a chicken egg, obtain an image signal of the egg shape, store threshold value related to the shape of the chicken egg, determine the sex of a fertilized unhatched egg by comparing and correlating the input image signal from the image-sensing apparatus to the stored threshold value and output that predicted sexing determination.

In this manner, the sex of the fertilized unhatched egg can be determined on the basis of generalized features of the shapes of the eggs for a given variety of chicken, and in the processes establishing threshold values for determining the sex of a fertilized chicken egg. Thus, the present invention provides a speedy, practical and reliable method and apparatus for determining the sex of a fertilized chicken egg, in which threshold value obtained by cataloging the various shapes of chicken eggs of any given variety of chicken and extracting the features in common to all such eggs is used to computer-Process the shape of any egg to be sexed.

With an apparatus having an image sensing means that uses a low-reflectance background (in other words, a background that easily absorbs light), the contrast (proportional brightness) between the surface of the bird egg and the background and the roundness becomes clear. Accordingly, the contour coordinates can be extracted accurately and accurate data can be obtained when scanning the screen, so the accuracy and reliability of the sexing of the egg is improved.

With an apparatus having a means of illuminating the surface of the rear of the egg as seen from the direction of image acquisition, both the sensed side and the rear side of the egg are illuminated and so the roundness of the contour of the egg as seen in the image of the egg becomes clear. By illuminating the egg so that the ridges in the egg are clear when seen from the camera, the contour coordinates can be extracted accurately and accurate data can be obtained when scanning the screen, so the accuracy and reliability sexing of the egg is improved.

With an apparatus having a downwardly concave and substantially egg-shaped stand for holding the egg, with the rim shaped so that it is smaller than the largest diameter of any such egg to be imaged and sexed, the surface of the egg contacts virtually the entire rim of the concavity, so the egg is positioned stably, the attitude (levelness) of the egg when placed on the stand can be adjusted easily and the accuracy of the attitude is improved.

With an apparatus having an inclined portion that slants toward an interior of the stand on an inner side of the rim of the stand in which a cushion is provided on a surface of the inclined portion, the cushion material closely contacts the surface of the egg over a large area and thus the egg can be even more stably positioned, the attitude (levelness) of the egg when placed on the stand can be adjusted easily and the accuracy of the attitude is further improved. Additionally, providing the cushion prevents the egg from being broken if the egg is placed in the concavity somewhat forcefully.

[First Embodiment]

A more detailed description will now be given of a first embodiment of the present invention as shown in the drawings.

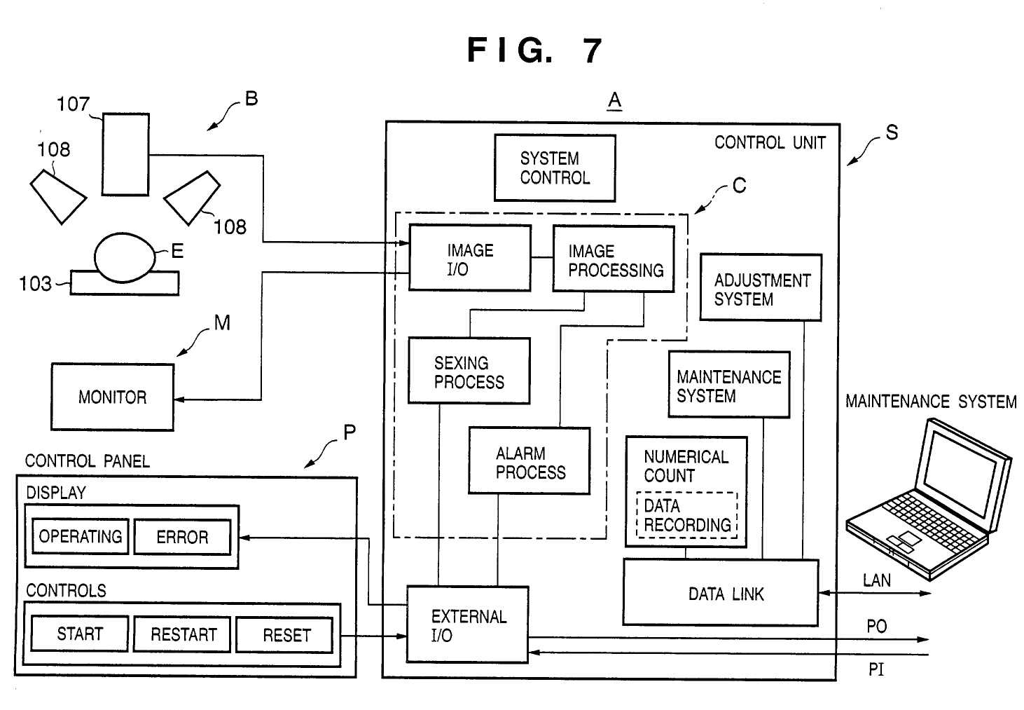

In order to facilitate a better understanding of the present invention, a brief description will first be given of an apparatus adapted for use with the present invention, with reference to FIGS. 7 through 12.

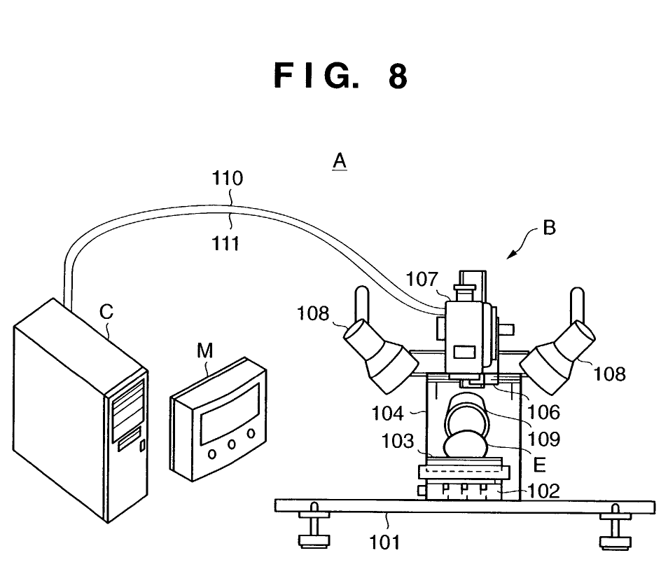

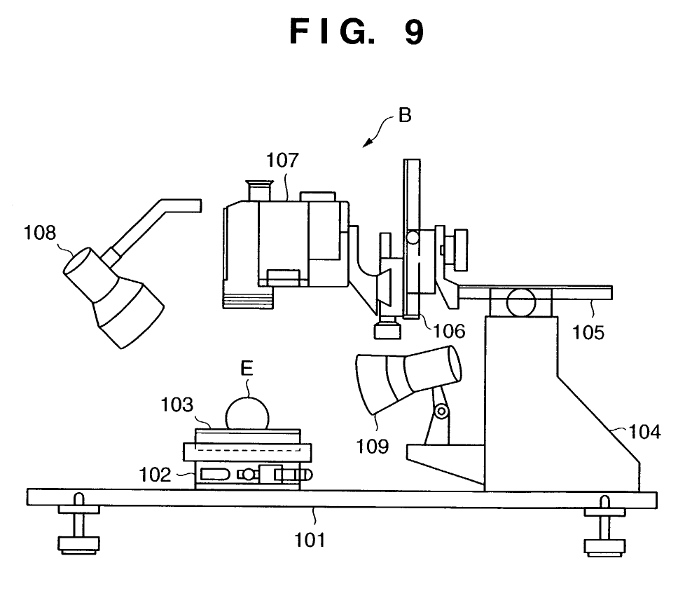

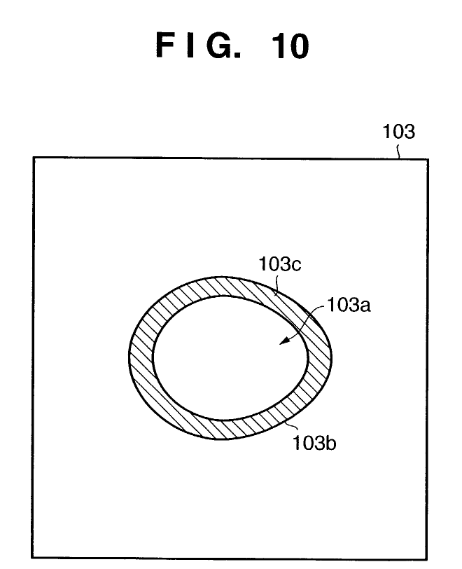

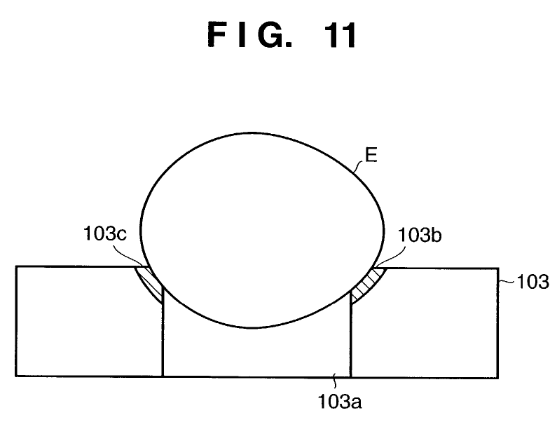

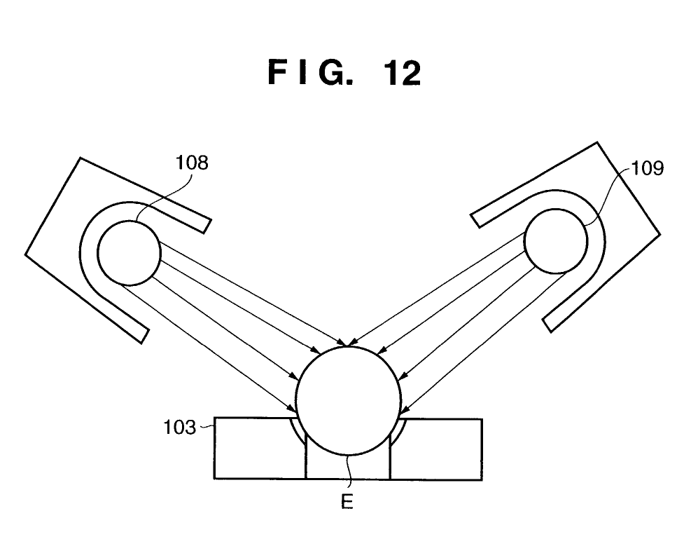

FIG. 7 is a diagram illustrating a sexing system including an apparatus for determining the sex of a fertilized chicken egg according to the present invention. FIG. 8 is a diagram showing an apparatus for determining the sex of a fertilized chicken egg and a front view of an image-sensing apparatus as part of the apparatus for determining the sex of a fertilized chicken egg. FIG. 9 is a lateral view of the image-sensing apparatus of the apparatus for determining the sex of a fertilized chicken egg shown in FIG. 8. FIG. 10 is a plan view of the egg stand of the image-sensing apparatus. FIG. 11 is a vertical cross-sectional view of the egg stand of the image-sensing apparatus. FIG. 12 is a diagram illustrating one method of illuminating a chicken egg using a lighting fixture of the image-sensing apparatus.

As shown in the drawings, a sexing system A is provided with an image-sensing apparatus B, a computer C and a monitor M. The image-sensing apparatus B is equipped with a measuring unit for measuring the shape of the egg and outputting the measurement data, and more particularly, is equipped with an image-acquisition unit for acquiring an image of the egg and outputting an image signal. The computer C is provided with a storage unit for storing threshold values for determining the sex of the eggs as correlated with the shape of the eggs, as well as a comparing unit for comparing the threshold values with data input from the measuring unit, and more particularly, is provided with a unit that determines the sex of the egg by comparing the threshold values with sexing values computed using sexing equations from an image signal input from the image-acquisition unit.

The sexing system A is further provided with a control unit S for controlling the entire system, the control unit S including a control panel P and a computer C. The control unit S controls an adjustment system and a maintenance system, and also controls a numerical quantity count system that counts the number of eggs sexed and stores the count as data. In addition, these systems are operatively connected to other computers via a LAN or the like. An image of an egg input to the computer C is subjected to image processing and sexing processing and output to the monitor M. The warning system outputs a warning signal when a interference or noise is present, in the form of dirt or dust attached to the image data, etc.

As shown in FIGS. 8 and 9, the image-sensing apparatus B is provided with a measuring mechanism retention platform 101 capable of horizontal adjustment. A camera retention platform 104 is provided at a rear side portion of the measuring mechanism retention platform 101. Camera positioning mechanisms 105, 106 capable of adjusting the position of the camera in three dimensions are provided atop the camera retention platform 104. A video camera 107 that faces downward is mounted on the camera positioning mechanism 106. An egg stand horizontal rotation adjustment mechanism 102 that can be rotated horizontally is provided at substantially the center of the measuring mechanism retention platform 101. An egg stand 103 is mounted on top of the egg stand horizontal rotation adjustment mechanism 102.

As shown in FIGS. 10 and 11, the surface of the egg stand 103 is painted a non-reflective black color. A downwardly concave nesting portion 103a is provided at the center of the stand 103. The center of the central nesting portion 103a is open toward the bottom. A rim part 103b of the central nesting portion 103a of the egg stand 103 is formed smaller than the largest part of the egg, though generally in the same shape as a chicken egg.

The surface of the rim part 103b slants downward toward the center of the stand 103. The angle of slant may be approximately 45 degrees. The surface of the slant is covered with a soft black felt 103c or some such similar material, so as to prevent damage to the eggs during handling.

The top of the rim part 103b is shaped to be generally 10 percent smaller than an egg, though the range of reduction is generally set between 5 percent and 15 percent. If the rate of reduction is less than 5 percent, the egg descends so low into the central nesting portion 103a of the egg stand 103 that it can be difficult to extract when attempting to measure the next egg. If the rate of reduction is more than 15 percent, the stability of the egg on the stand is less than ideal.

A forward lighting fixture 108 is positioned above and at an angle to the egg stand 103, with a rear lighting fixture 109 positioned at the rear of and at an angle to the egg stand 103.

As shown in FIG. 12, the forward lighting fixture 108 basically illuminates the front of a chicken egg E and the rear lighting fixture 109 basically illuminates the rear of the chicken egg E. The lighting fixtures 108, 109 also illuminate a back side of the egg hidden by the curved portion of the egg when viewed from above, which serves to heighten and clarify the contrast between the curvature of the egg E and the surface of the egg stand 103. Clarifying the contrast allows the coordinates of the profile of the egg based on the acquired image of the egg to be extracted reliably. In addition, it should be noted that reference numeral 110 denotes an analog video signal line, reference numeral 111 denotes a video digital data signal line.

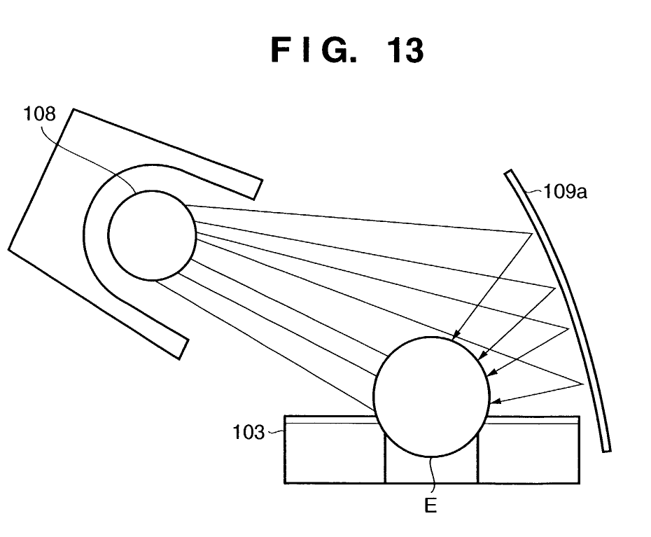

FIG. 13 is a diagram illustrating another method of illuminating a chicken egg using a lighting fixture of the image-sensing apparatus. As shown in the diagram, a concave mirror 109a is positioned to the rear of the egg stand 103. The concave mirror 109a reflects light from the lighting fixtures 108 to the front so as to illuminate the rear of the chicken egg E.

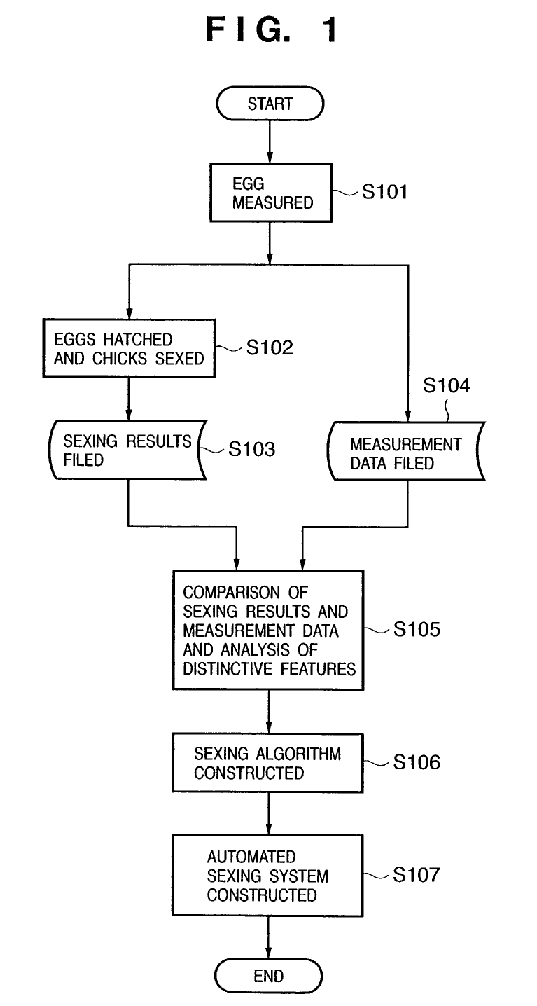

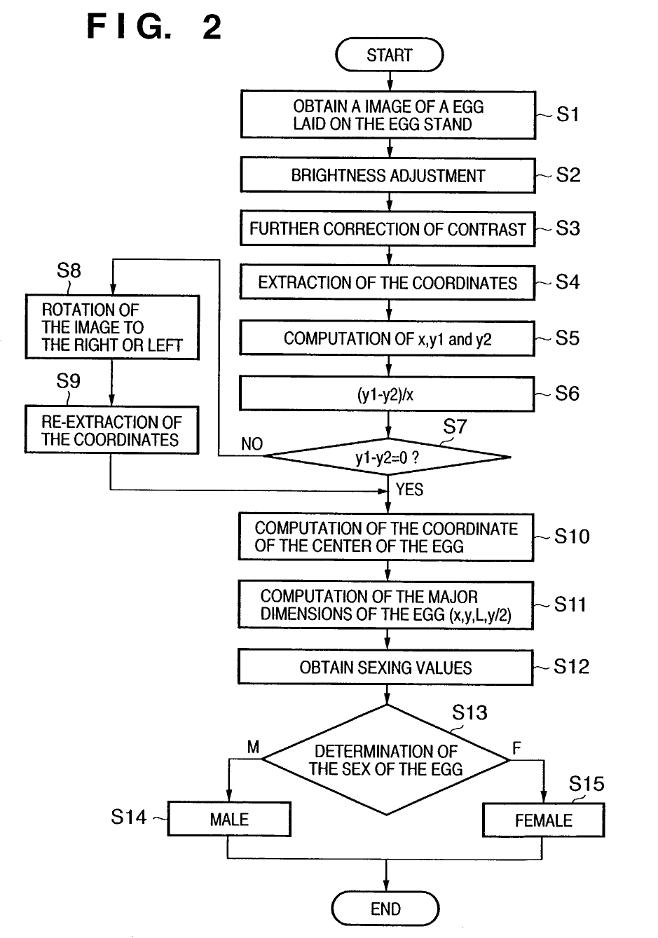

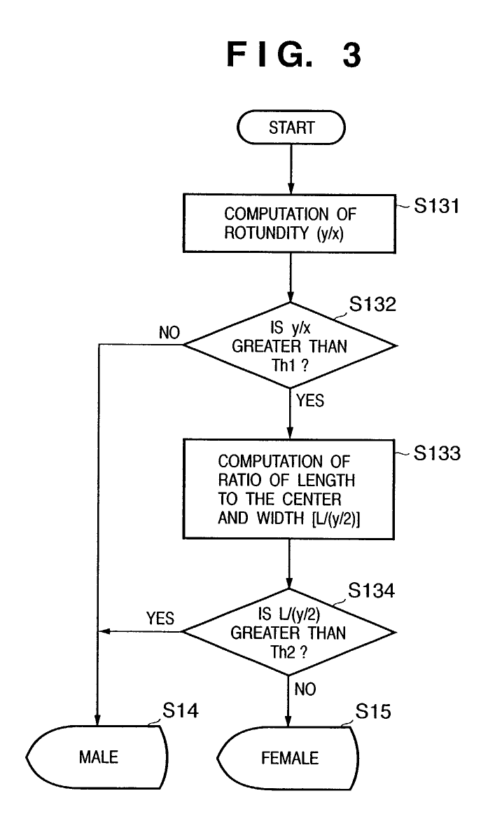

FIG. 1 is a flow chart showing steps in the construction of an algorithm for determining the sex of an unhatched chicken egg according to the present invention. FIG. 2 is a flow chart showing an example of the method for determining the sex of a fertilized chicken egg according to the present invention. FIG. 3 is a flow chart showing specific steps in the sex determination portion of the process shown in FIG. 2. The sexing of a chicken egg, that is, determining the sex of the fertilized chicken egg, is carried out in the following manner.

First, as shown in FIG. 1, an algorithm to be used in determining the sex of the unhatched chick in the egg is constructed (S107). Through a series of steps S1 through S12 such as those described below, data is collected on the shape of fertilized eggs from the same variety of chicken as that which produced the egg to be sexed using the required number of images (S101, S104), the data on shape so collected is then checked against the results of a manual determination (S105) by an expert of the sex of the chicks after hatching from the eggs (S102, S103), and threshold values for determining the sex of the unhatched egg are established using predetermined sexing equations that correlate the shape of the egg with the sex of the hatchling (S106).

A description will now be given of steps in a sexing process, with reference to FIG. 2.

In a step S1, a chicken egg E to be sexed is laid on its side so that it rests horizontally in the concave nesting portion 103a provided at the center of the egg stand 103. An image of the egg E is then acquired from above by the digital video camera 107 and the output therefrom is read into the computer C.

In a step S2, the brightness of the image is adjusted to sharpen the contrast between the contour of the egg E and the egg stand 103 that is the background.

In a step S3, the image is then corrected to further intensify the contrast between the egg and the background.

In a step S4, dust and other debris that could create interference is removed from the surface of the egg or the egg stand 103 that forms the background and the entire surface of the image is scanned. While checking the contour of the egg, that is, reading the coordinates of points of sharp contrast, the apparatus detects and stores in a memory unit (1) a coordinate of narrow end, (2) a coordinate of blunt end, (3) a coordinate of lower end and (4) a coordinate of upper end.

In a step S5, from the above-described coordinates (1), (2), (3) and (4), the apparatus computes the maximum length x of the egg E, a widthwise direction coordinate of a blunt end y1 and of a narrow end y2 (neither y1 nor y2 appearing in the drawings).

In a step S6, the apparatus performs calculation of a horizontal displacement (y1−y2)/x and a correction angle.

In a step S7, the apparatus determines whether or not y1−y2=0. If y1−y2=0, then the process proceeds to a step 10. If y1−y2≠0, then the process proceeds to a step 8.

In step S8, if y1−y2 is a positive result, then the image is rotated to the right by an amount equal to the angle computed in step 6. If y1−y2 is a negative result, then the image is rotated to the left by an amount equal to the angle computed in step 6.

In a step S9, the entire surface of the angle-corrected image is scanned. While checking the contour of the egg E, that is, while reading the coordinates of the points of sharp contrast, the apparatus once again detects and stores in a memory unit (1) a narrow end coordinate, (2) a blunt end coordinate, (3) a lower end coordinate and (4) a upper end coordinate.

In step S10, the apparatus computes the center e of the image of the egg E.

In a step S11, the apparatus computes the major dimensions of the egg E, that is, the maximum length x, the maximum width y, the a length of wide side L, and a width y/2 which is a length between a upper end d and the center e.

In a step S12, sexing values are obtained using sexing equations (y/x) and (L/(y/2)).

In a step S13, the obtained sexing values are compared to threshold values to make a determination as to the sex of the egg.

A more detailed description will now be given of steps S12 and S13, with reference to FIG. 3.

As described above, FIG. 3 is a flow chart showing specific steps in the sex determination portion of the process shown in FIG. 2. In a step S131, the apparatus performs (y/x) calculations to obtain the sexing values as to the sex of the fertilized unhatched eggs. In a step S132, the obtained sexing values are compared to threshold value Th1 to make a provisional determination as to the sex of the egg. Eggs found to be male are sexed as male and are male-displayed 14.

In step S133, eggs not found to be male in step S132 are further analyzed using the sexing equation (L/(y/2)) to obtain sexing values as to the sex of the fertilized unhatched egg. In a step S134, the obtained sexing values are compared to threshold value Th2 to make a final determination as to the sex of the egg. Eggs found to be male are sexed as male and are male-displayed 14. Eggs not found to be male in either steps S131-S132 or steps S133-S134 are sexed as female and are female-displayed 15.

[Second Embodiment]

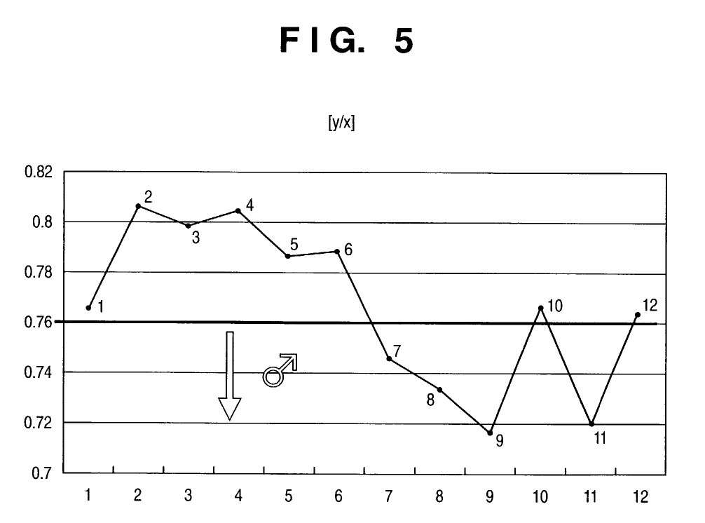

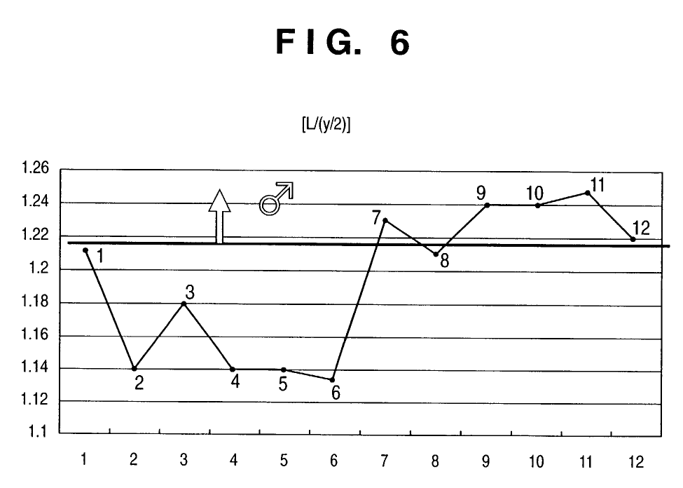

A description will now be given of a second embodiment of the present invention, with reference to FIGS. 5 and 6.

FIG. 5 is a graph plotting results of calculations performed using a sexing equation (y/x) on the chicken eggs to be sexed. FIG. 6 is a graph plotting results of calculations performed using a sexing equation (L/(y/2)) on the chicken eggs to be sexed.

In a second embodiment of the present invention, a dozen fertilized eggs of the White Leghorn variety of chicken are prepared, the numbers of the chicken eggs in the drawings are divided by a sexing expert into male and female upon examination after hatching. For ease and clarity of explanation, Nos. 1-6 are classed as female and Nos. 7-12 are classed as male.

Sexing values for each of the fertilized chicken eggs Nos. 1-12 were obtained using steps S1-S12 described above. The threshold values used to sex the unhatched eggs are computed to be 0.76 using the sexing equation (y/x) of FIG. 5 and 1.22 using the sexing equation (L/(y/2)) of FIG. 6. These threshold values sometimes change as more data is collected or depending on the variety of chicken.

As shown in FIG. 5, the results of computations performed using the sexing equation (y/x) for the chicken eggs to be sexed are depicted in graph form. As indicated, sexing values are computed for each chicken egg using the sexing equation (y/x), with results below the threshold value Th1 of 0.76 being classified as male. Thus. Egg Nos. 7, 8, 9 and 11 were found to be male. It will be noted that egg Nos. 10 and 12, which were actually male, were not deemed to be males according to the sexing equation (y/x) used in FIG. 5.

As shown in FIG. 6, the results of computations performed using the sexing equation (L/(y/x)) for the chicken eggs to be sexed are depicted in graph form. Computations are performed on egg Nos. 1-6. 10 and 12 deemed not to be males using the sexing equation (y/x) and sexing values obtained. Those sexing values exceeding the threshold Th2 of 1.22 are deemed to be males, and by this test egg Nos. 10 and 12 are deemed to be males.

Ultimately, the eggs deemed to be not male are egg Nos. 1-6, and these eggs are deemed to be female, that is, are sexed as female. It should be noted that these sexing results coincide with the sexes of the chicks as found after hatching, indicating the validity of the present invention. It should be noted that the same results are obtained if the order of the tests is reversed, that is, if the sexing equation (L/(y/2)) is used prior to the use of the sexing equation (y/x).

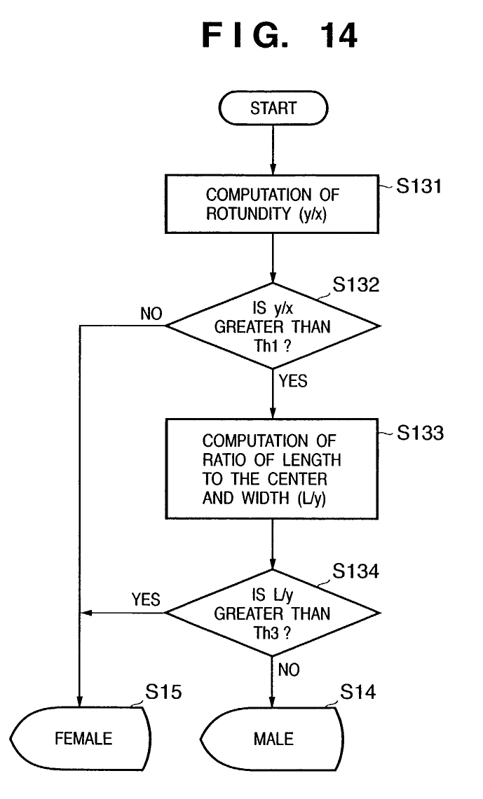

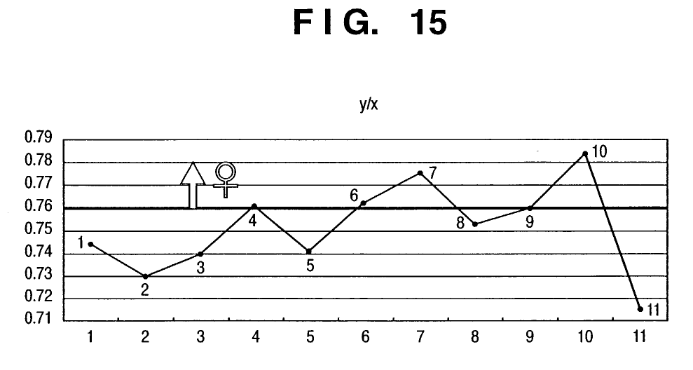

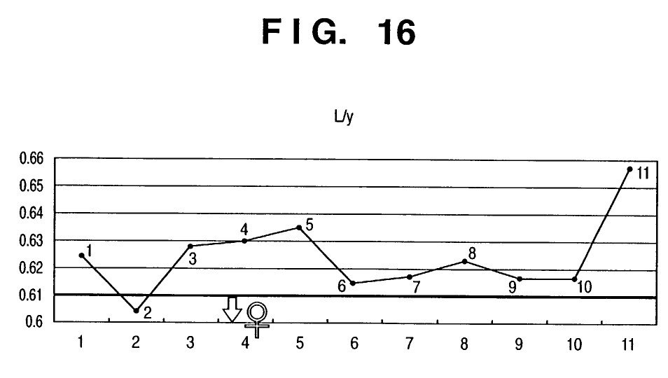

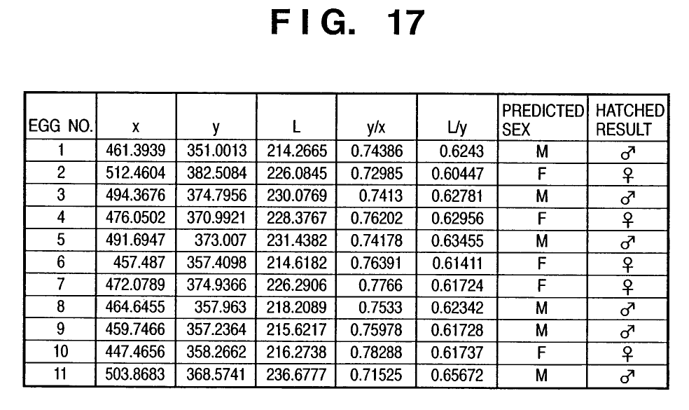

FIG. 14 is a flow chart showing another example of the method for determining the sex of a fertilized chicken egg, showing specific steps in the sex determination portion of a process similar to the process shown in FIG. 1, in which, as with the flow chart depicted in FIG. 2, those steps having to do with actually determining the sex of the fertilized unhatched eggs are described in detail. FIG. 15 is a graph plotting results of calculations performed using a sexing equation (y/x) on the chicken eggs to be sexed. FIG. 16 is a graph plotting results of calculations performed using a sexing equation (L/y) on the chicken eggs to be sexed. FIG. 17 is a table showing the sex of the chicks hatched from the measured eggs, together with the results of calculations performed using the sexing equations.

A detailed description will now be given of steps S13 and S14 of another sexing method, with reference to FIG. 14. It should be noted that in this case, in step S12, the major dimensions of the chicken egg (that is, maximum length x, maximum width y, and length L) is computed. Then, in a step S131, the computation (y/x) is performed and sexing values are obtained. In a step S132, the sexing values are then compared to a threshold value Th1, with values exceeding the threshold being deemed females and are female-displayed 15.

In a step 133, those eggs not found to be female in step S132 are then subjected to computations using the sexing equation (L/y) and sexing values are obtained. In a step S134, the sexing values are then compared to a threshold value Th3, with values exceeding the threshold being deemed females and are female-displayed 15. Eggs not found to be female in either steps S131-S132 or steps S133-S134 are sexed as male and are male-displayed 14.

[Third Embodiment]

A description will now be given of a third embodiment of the present invention, with reference to FIGS. 15, 16 and 17.

In the third embodiment of the present invention, 11 fertilized eggs of the White Leghorn variety of chicken are prepared and sexing values are obtained using the above-described processual steps. It should be noted that the established threshold values (that is, the threshold values th1 and Th3) for determining the sex of the fertilized egg are 0.76 as computed using the sexing equation (y/x) of FIG. 15 and 0.61 as computed using the sexing equation (L/y) of FIG. 16. These threshold values may change as more data is collected and may vary according to the variety of chicken.

As shown in FIG. 15, computed values (that is, preliminary sexing values) for each of the chicken eggs as computed using the formula (y/x, that are greater than 0.76 are deemed to be female. In the present example, egg Nos. 4, 6, 7 and 10 are classed as female. It should be noted that egg No. 2, which is actually also female, is not deemed to be female by the results of the computations performed using the given sexing equation described above.

As shown in FIG. 16, the eggs not deemed to be female using the equation y/x, that is, in this case, egg Nos. 1, 2, 3, 5, 8, 9 and 11, are re-examined using the sexing equation (L/y) in order to obtain sexing values. Eggs for which the computed sexing value is smaller than 0.61 are deemed to be female. In the present example, egg No. 2 is deemed to be female by this further equation.

Thus, the eggs ultimately not classed as female are Nos. 1, 3, 5, 8, 9 and 11. In the present example, chicken eggs Nos. 1, 3, 5, 8, 9 and 11 are deemed to be male. As shown in FIG. 17, these results are the same as those found by having a professional manually determine the sex of the chicks after hatching, so the accuracy of the present method for determining the sex of a fertilized chicken egg would appear to be very high, indeed 100 percent. It should be noted that the same results are obtained if the order of application of the sexing equations to the eggs is reversed, that is, if the use of the (L/y) sexing equation precedes the use of the (y/x) equation.

It should be noted that the terms and expressions used in this specification are intended to be descriptive only, are not necessarily limiting, and do not eliminate equivalent terms and expressions. Similarly, the present invention is not limited to the embodiments shown in the drawings. Rather, it is to be understood that the present invention is not limited to the above embodiments, and various changes and modifications can be made within the spirit and scope of the present invention.

As described above, the present invention provides a speedy, practical and reliable method and apparatus for determining the sex of a fertilized chicken egg, in which threshold value obtained by cataloging the various shapes of chicken eggs of any given variety of chicken and extracting the features in common to all such eggs is used to computer-Process the shape of any egg to be sexed.

As many apparently widely different embodiments of the present invention can be made without departing from the spirit and scope thereof, it is to be understood that the invention is not limited to the specific embodiments thereof except as defined in the appended claims.

What is claimed is:

1. A method for determining the sex of a fertilized egg, comprising the steps of:
measuring shapes of a plurality of chicken eggs to obtain measurement data;
hatching the measured eggs and determining the sex of the hatchlings;
correlating the measurement data on the shape of the eggs with the sex of the chickens hatched from the measured eggs to obtain threshold value that correlates egg shape to the sex of the unhatched chick contained therein; and
measuring the shapes of eggs laid by the same variety of chicken as that which laid the plurality of eggs and determining the sex of an unhatched chick in any of such eggs using the threshold value.

2. A method for determining the sex of a fertilized egg, comprising the steps of:
measuring shapes of a plurality of chicken eggs to obtain measurement data;
hatching the measured eggs and determining the sex of the hatchlings;
correlating the measurement data on the shape of the eggs with the sex of the chickens hatched from the measured eggs to obtain threshold value that correlates egg shape to the sex of the unhatched chick contained therein; and
measuring the shapes of eggs laid by the same variety of chicken as that which laid the plurality of eggs and determining the sex of an unhatched chick in any of such eggs using the threshold value.

3. A method for determining the sex of a fertilized egg by measuring a shape of the egg and referring to threshold value that correlates egg shape to the sex of the unhatched chick contained therein, wherein the threshold value is obtained from the shape data obtained by measuring the shapes of a plurality of eggs from the same variety of chicken and a data of the sex of the chicks determined after they are hatched from the plurality of eggs.

4. A method for determining the sex of a fertilized egg, comprising the steps of:
a threshold value obtaining step of obtaining a threshold value by correlating measurement data on the shape of the eggs with the sex of the chicks hatched from the measured eggs;
a sexing value obtaining step of obtaining a sexing value based on measurement data on the shape of an egg to be sexed; and
a comparing step of comparing the threshold value and the sexing value to determine the sex of the unhatched chick.

5. A method for determining the sex of a fertilized egg, comprising the steps of:
a threshold value obtaining step of obtaining a threshold value by correlating measurement data of the length and width of a plurality of eggs with the sex of the chicks hatched from the measured eggs;
a sexing value obtaining step of obtaining a sexing value by using measurement data or calculated data of the length and width of an egg to be sexed; and
a comparing step of comparing the threshold value and the sexing value to determine the sex of the unhatched chick.

6. A method for determining the sex of a fertilized egg, comprising the steps of:
a threshold value obtaining step of obtaining threshold value by correlating measurement data of the length, width and center of a plurality of eggs with the sex of the chicks hatched from the measured eggs;
a sexing value obtaining step of obtaining a sexing value by using measurement data or calculated data of the length, width and center of an egg to be sexed; and
a comparing step of comparing the threshold value and the sexing value to determine the sex of the unhatched chick.

7. A method for determining the sex of a fertilized egg, comprising the steps of:
a threshold value obtaining step of obtaining a threshold value by correlating data of the length, width and center of a plurality of eggs calculated based on an image data of the plurality of eggs with the sex of the chicks hatched from the plurality of eggs;
a sexing value obtaining step of obtaining a sexing value by using data of the length, width and center of an egg to be sexed calculated based on the image data of the egg to be sexed; and
a comparing step of comparing the threshold value and the sexing value to determine the sex of the unhatched chick.

8. The method according to claim 7, further comprising a tilt correction step of correcting a tilt of the image of the egg to be measured or sexed; and
a calculating step of calculating data of the length, width and center of the egg to be measured or sexed based on the corrected image of the egg to be measured or sexed.

9. A method for determining the sex of a fertilized egg, comprising the steps of:
acquiring an image of an egg to be sexed using an image-sensing apparatus and outputting the acquired image to an information processing apparatus;
extracting coordinates of a contour of the egg from the output image;
computing a length and width of the egg from the extracted coordinates;
computing an angle of tilt of the image from the coordinates of the computed length and width of the egg and correcting the image for the angle of tilt;
extracting coordinates of a contour of the egg from the corrected image of the egg;
computing coordinates of a length, width and center of the egg from the extracted coordinates of the contour;
computing a sexing value for the purpose of determining the sex of the fertilized egg according to a predetermined sexing equation using the coordinates of the length, width and center of the egg; and
determining the sex of the fertilized egg by comparing the computed sexing value to an established threshold value for determining the sex of the fertilized egg.

10. The method according to claim 9, wherein the sexing equation is an equation expressing a proportion between measured length and measured width of an egg to be sexed.

11. The method according to claim 9, wherein the sexing equation is an equation expressing a proportion between a measured width of the egg and measured length from a center to a blunt end of the egg.

12. The method according to claim 9, wherein the sexing equation comprises:
a first equation expressing a proportion between measured length and measured width of an egg to be sexed; and
a second equation expressing a proportion between measured width and measured length from a center to a blunt end of the egg.

13. The method according to claim 12, wherein the sex of the unhatched chicken egg is ultimately determined to be female when the results of comparisons of both the first equation and the second equation with their respective threshold values indicate that the sex of the unhatched chick in the egg is female.

14. The method according to claim 12, wherein the sex of the unhatched chicken egg is ultimately determined to be female when the results of comparisons of at least one of the first equation and the second equation with their respective threshold values indicate that the sex of the unhatched chick in the egg is female.

15. The method according to claim 7, wherein:
the image of the egg to be measured or sexed includes maximum and minimum coordinates indicating the length of the egg and maximum and minimum coordinates indicating the width of the egg; and
a scan of the image of the egg to be measured or sexed includes a portion including at least 3 coordinates when the image of the egg to be measured or sexed is scanned,
the three coordinates including at least the maximum and minimum coordinates indicating the length of the egg.

16. A method for determining the sex of a fertilized egg, comprising the steps of:
obtaining values for determining the sex of a plurality of eggs from the shape of the plurality of eggs from a plurality of hens of a given variety of chicken;
generating threshold values for sexing a chick by using said values;
comparing said values from the same variety of chicken with the threshold values to determine the sex of the unhatched chick.

17. A method for determining the sex of a fertilized egg, comprising the steps of:
obtaining values for determining the sex of a plurality of eggs from a plurality of hens of a given variety of chicken based on the shape of the plurality of eggs;
inputting the shape data of eggs to be sexed from the same variety of chicken to the sexing unit which has threshold values for sexing a chick generated by using of said values; and
comparing the shape data with the threshold values to determine the sex of the unhatched chicken egg.

18. An apparatus for determining the sex of a fertilized egg, the apparatus comprising:
a measuring unit for measuring shapes of a plurality of chicken eggs and output measurement data;
a storage unit for storing threshold values correlating the measurement data on the shape of the eggs with the sex of the chickens hatched from the measured eggs; and
a comparing unit for comparing the measurement data input from the measurement means with the threshold values in order to determine the sex of an unhatched chick in any of such eggs; and
an output unit for outputting the determination of the sex of the unhatched chick resulting from the comparison of the measurement data with the threshold values.

19. An apparatus for determining the sex of a fertilized egg, the apparatus comprising:
an image-sensing unit for sensing the shape of an egg to be measured and sexed and outputting an image signal thereof;
a storage unit for storing threshold values correlating the shape of the eggs with the sex of the chickens hatched therefrom;
a sexing unit for determining the sex of an unhatched chick in any of such eggs by comparing the image signal input from the image-sensing unit with the threshold values; and
an output unit for outputting the determination of the sex of the unhatched chick resulting from the comparison of the image signal with the threshold values.

20. The apparatus according to claim 19, wherein the image-sensing unit uses a low-reflectance background.

21. The apparatus according to claim 19, further comprising a lighting unit for illuminating a surface of the egg opposite the surface of the egg facing the image-sensing unit.

22. The apparatus according to claim 19, further comprising a downwardly concave and substantially egg-shaped stand for holding the egg, the pedestal having an outer rim smaller than the largest diameter of any such egg to be imaged and sexed.

23. The apparatus according to claim 22, wherein:
an inclined portion that slants toward an interior of the stand is provided on an inner side of the rim of the stand; and
a cushion is provided on a surface of the inclined portion.
